(19) United States
(12) Patent Application Publication
(10) Pub. No.: US 2021/0267332 A1
(43) Pub. Date: Sep. 2, 2021

(54) PERSONAL SANITATION TRAVEL KIT AND COMPONENTS THEREOF

(71) Applicant: Regina Arlene Gies, Dawson, IL (US)

(72) Inventor: Regina Arlene Gies, Dawson, IL (US)

(21) Appl. No.: 17/249,380

(22) Filed: Mar. 1, 2021

Related U.S. Application Data
(60) Provisional application No. 62/983,057, filed on Feb. 28, 2020.

(51) Int. Cl. A45C 11/00; A47C 7/38; B60N 2/60; A47C 7/62; A47G 11/00; A61F 9/04; A61F 11/08

(52) U.S. Cl. CPC A45C 11/008; A47C 7/383; B60N 2/60; A47C 7/386; A47K 10/22; A61F 9/04; A61F 11/08; A45C 2011/007; A45C 2200/10; A47G 11/003

(57) ABSTRACT

A disinfectant and antibacterial travel kit for passenger travel and airlines. To protect person from getting sick from the germs in the airplane recirculating air and all the germs from previous passenger left on the seatbelt, seat, tray table, public airplane bathroom. Also, the kit may be used on all forms of public transportation. Trains, subway, buses, carpool etc. The kit according to the present invention helps travelers stay healthy while using public transportation.

### CROSS-REFERENCE TO RELATED APPLICATION

This application claims the benefit of priority of U.S. provisional application No. 62/983,057, filed Feb. 28, 2020, the contents of which are herein incorporated by reference.

### BACKGROUND OF THE INVENTION

The present invention relates to personal sanitation items, and more particularly to personal sanitation items for use in travel situations.

Movement via public transportation presents numerous sanitation issues to the traveler. The lack of cleanliness of the transport platform, such as a bus, a train, or an airplane can expose the traveler to a wide variety of germs. In the case of airline transport platforms, these germs may have migrated internationally either through the aircraft traveling internationally, or exposure to travelers that have.

For travelers with compromised immune systems, such as those undergoing cancer treatments, the risks of exposure to these germs is particularly acute. While such patients would typically not travel, access to specialized medical centers for treatment may make travel by carriers necessary. In these cases, the traveler is unable to rely on the cleanliness of a particular carrier for providing a clean occupant space while traveling.

In my own case, after completing initial chemotherapy treatments, I found myself in need of traveling to attend my mother's funeral. As a nurse with over 25 years of experience in both hospital and clinical settings, I was surprised to discover that while various individual sanitation items exist, there are no personal sanitation travel kits that provide travelers with a sufficient variety of cleaning items for disinfecting occupant space in a transport platform. I also discovered that some personal sanitation items are not well adapted to the travel environment.

As can be seen, there is a need for an improved personal sanitation travel kit and components thereof, that are particularly useful for patients with compromised immune systems that need to travel, such as to attend family occasions, attend needed treatments at remote medical centers, and for some to travel to bucket list destinations.

### SUMMARY OF THE INVENTION

In one aspect of the present invention, a personal sanitation travel kit is disclosed. The personal sanitation travel kit includes a carrier having at least one sidewall defining an interior compartment. A top opening provides access to the interior compartment. At least one handle is coupled to a top end of at least one sidewall. A plurality of sanitation and personal protective items are removably contained within the interior compartment. The plurality of sanitation and personal protective items including, a packeted disinfecting wipe having a quantity of wipes to disinfect an occupant seating space within a transport vehicle, a medical mask, and a pair of latex free gloves.

In some embodiments, the at least one sidewall is made of a transparent material to permit viewing of the plurality of sanitation and personal protective items within the interior compartment.

In some embodiments, the personal sanitation travel kit includes a packet of hand sanitizing wipes and a 2-3 Oz. bottle of a hand sanitizer liquid. A tray cover dimensioned to cover a passenger tray in the occupant space of the transport vehicle; a headrest cover dimensioned to cover head rest of an occupant seat of the transport vehicle; and a mini roll of multi-ply toilet paper may also be included.

In some embodiments, an eye mask is provided to facilitate sleep and to prevent drying or contamination through the eyes from a ventilation duct directed in the occupant space. A pair of ear plugs configured for removable insertion into an ear canal of the passenger to reduce sound levels within the travel environment of the transport vehicle.

In some embodiments, an inflatable neck pillow to facilitate sleep and discomfort while confined to the occupant seating space of the transport vehicle is provided. The inflatable neck pillow having an inflatable U-shaped bladder, a removable cover having an inner space dimensioned contain the inflatable U-shaped bladder. The inflatable neck pillow further may also include an access slot in the removable cover that is dimensioned to receive the inflatable U-shaped bladder within the inner space. A first cooperating fastener is provided to selectively open and close the access slot. A second cooperating fastener at a terminal end the cover is operable to secure the inflatable neck pillow about a neck of the passenger.

In yet other embodiments, the mini roll of multi-ply toilet paper further a compressed roll of the multi-ply toilet paper wrapped without an inner core insert, with the multi-ply toilet paper wrapped at a minimal radius. The minimal radius is preferably a negligible radius.

These and other features, aspects and advantages of the present invention will become better understood with reference to the following drawings, description and claims.

### BRIEF DESCRIPTION OF THE DRAWINGS

FIG. 1 is a front perspective view of a personal sanitation travel kit.

FIG. 2 is an exploded perspective view of the components of the personal sanitation travel kit.

FIG. 3 is a detail exploded perspective view of the pillow component of the personal sanitation travel kit.

### DETAILED DESCRIPTION

The following detailed description is of the best currently contemplated modes of carrying out exemplary embodiments of the invention. The description is not to be taken in a limiting sense but is made merely for the purpose of illustrating the general principles of the invention.

Broadly, embodiments of the present invention provide a system, method, and apparatus for providing a personal sanitation kit for travelers. The personal sanitation travel kit allows the traveler to sanitize elements of the travel environment encountered by a traveler to reduce the risk of contracting illnesses while traveling.

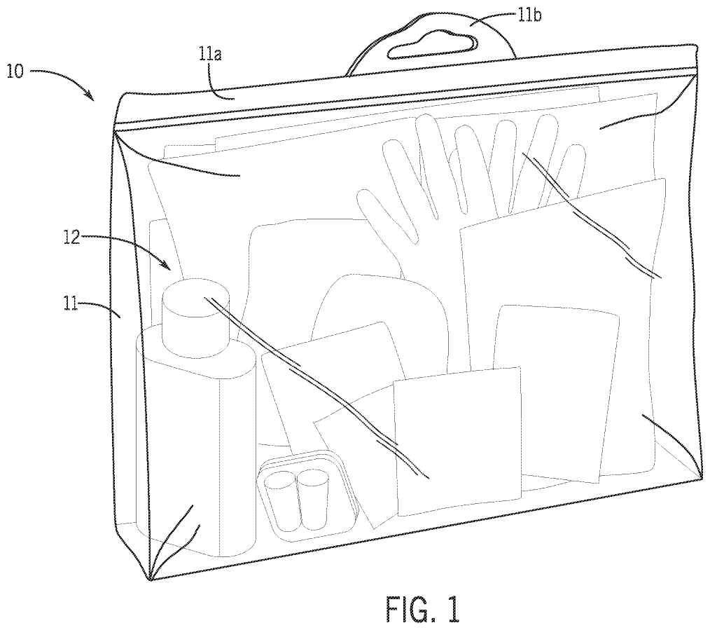

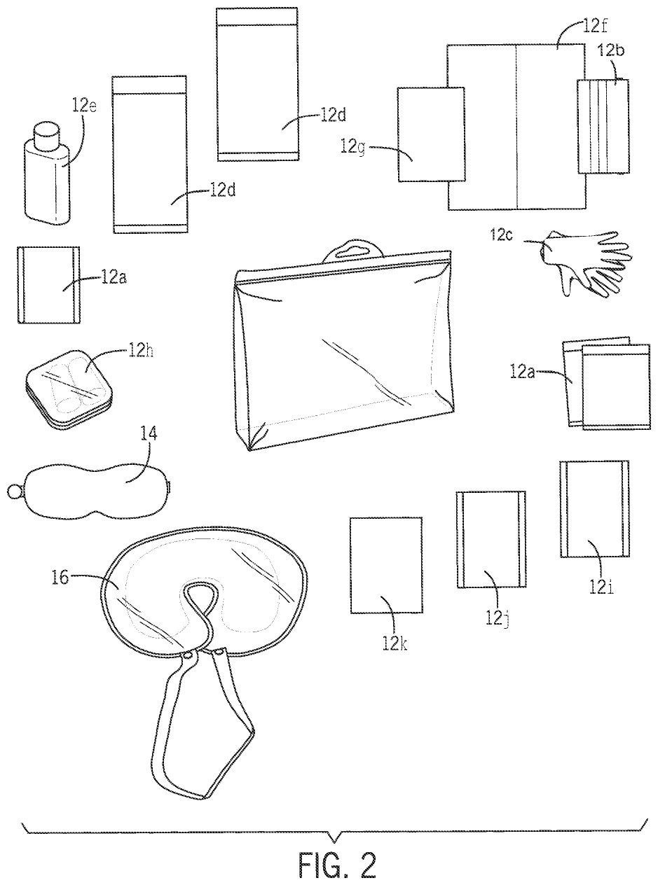

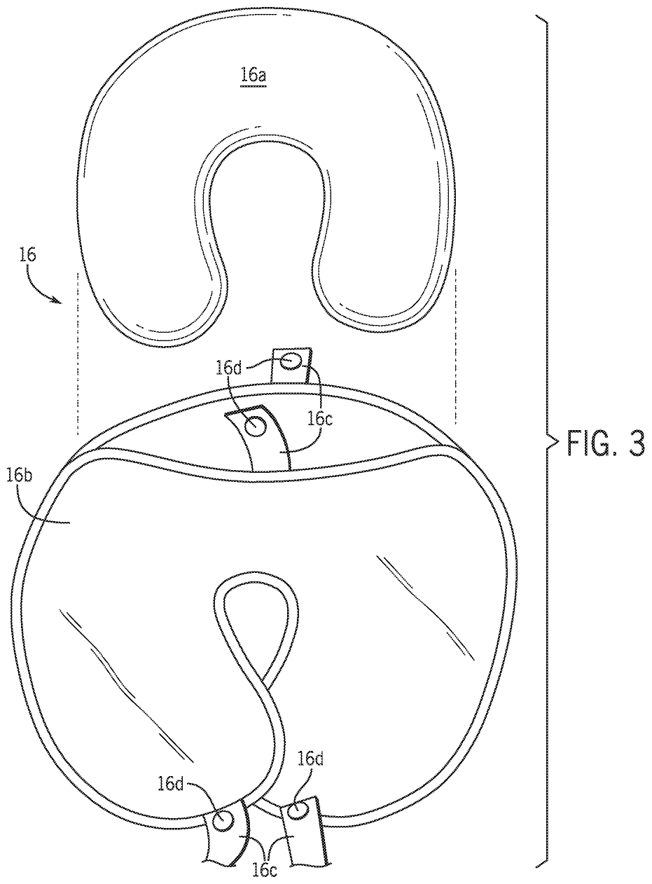

As seen in reference to the drawings of FIGS. 1-3, the personal sanitation travel kit 10 according to aspects of the invention includes a carrier 11 to contain a plurality of sanitation and personal protective items 12 while traveling. The carrier 11 includes a small portable container 11 having handles 11 b for carriage of the items and has a clear outer layer to permit viewing of the sanitation items contained therein to facilitate passage at security checkpoints encountered while traveling. A top opening 11 a is provided to access an interior compartment of the carrier 11.

The components 12 of the personal sanitation travel kit 10 may be configured for a particular mode of travel and may also include basic and premium kits selected according to a traveler's needs. A basic kit may include: a packeted disinfecting wipe 12a, such as a Lysol® wipes; a medical mask 12b, and a pair of latex free gloves 12c. The basic kit is suited to permit the traveler to protect themselves in the immediate vicinity of their transport pace, or seat. The gloves 12c permit the traveler to touch areas in the transport space. The medical mask 12b permits the traveler to protect themselves from airborne hazards. The disinfecting wipes 12a permit the user to disinfect the surface areas of their transport space. A plurality of disinfecting wipes 12a may be included to permit the traveler to disinfect surfaces within a toilet facility on the transport platform or in a terminal area for the transport platform.

An Intermediate Kit may include the components of the basic kit, and duplicate components thereof. The intermediate kit may also include a packet of hand sanitizing wipes 12d, such as PurellI® wipes , a 2-3 Oz. bottle of hand sanitizer 12e, such as Germx® to permit the traveler to sanitize their hands and skin while in the travel environment.

A Deluxe Kit may include the components of the basic and intermediate kits. The Deluxe Kit may also include: a tray cover 12f, a headrest cover 12g, a mini roll of 4 ply toilet paper 12h & optional bag to hold it. An eye mask 14 may also be provided to facilitate sleep and also to prevent drying or contamination through the eyes from a ventilation duct in the transport space. A pair of ear plugs may be provided to reduce sound levels within the travel environment. The ear plugs may include a case to maintain the ear plugs in a clean condition while not in use.

The Deluxe Kit may also include an inflatable neck pillow 16 to facilitate sleep and discomfort while confined to the transport space. The inflatable neck pillow 16 has an inflatable U-shaped bladder 16a, a removable cover 16b having an inner space dimensioned contain the inflatable U-shaped bladder 16a. An access slot is provided along a base of the U-shaped cover 16b and dimensioned to receive the inflatable U-shaped bladder 16a within the inner space. A first cooperating fastener 16d is carried on a tab 16c that are configured to selectively open and close the access slot. A second cooperating fastener 16d may be provided at a terminal end the cover 16b. The second cooperating fastener 16d is operable to secure the inflatable neck pillow 16 about a neck of the passenger.

The Deluxe Kit may also include urogenital hygiene items, such as a male wipe 12i or a female wipe 12j for toilet use, a toilet seat protective cover 12k, and additional skin sanitizer wipes 12d.

The toilet paper rolls 14 according to aspects of the invention provide a readily available source of paper that is maintained by the traveler in a clean environment while traveling. The toilet paper may be fabricated of a 4 ply bathroom tissue that is wound without, or on a core with a substantially reduced diameter in order to limit the space required to provide a suitable length or quantity of the bathroom tissue for the user.

It should be understood, of course, that the foregoing relates to exemplary embodiments of the invention and that modifications may be made without departing from the spirit and scope of the invention.

What is claimed is:

1. A personal sanitation travel kit, comprising:
a carrier having at least one sidewall defining an interior compartment, a top opening to provide access to the interior compartment;
at least one handle coupled to a top end of at least one sidewall;
a plurality of sanitation and personal protective items removably contained within the interior compartment, the plurality of sanitation and personal protective items comprising, a packeted disinfecting wipe having a quantity of wipes to disinfect an occupant seating space within a transport vehicle, a medical mask, and a pair of latex free gloves.

2. The personal sanitation travel kit of claim 1, wherein the at least one sidewall is made of a transparent material to permit viewing of the plurality of sanitation and personal protective items within the interior compartment.

3. The personal sanitation travel kit of claim 2, further comprising:
a packet of hand sanitizing wipes, and a 2-3 Oz. bottle of a hand sanitizer liquid.

4. The personal sanitation travel kit of claim 3, further comprising:
a tray cover dimensioned to cover a passenger tray in the occupant space of the transport vehicle;
a headrest cover dimensioned to cover head rest of an occupant seat of the transport vehicle; and
a mini roll of multi-ply toilet paper.

5. The personal sanitation travel kit of claim 4, further comprising:
an eye mask to facilitate sleep and to prevent drying or contamination through the eyes from a ventilation duct directed in the occupant space.

6. The personal sanitation travel kit of claim 4, further comprising:
a pair of ear plugs configured for removable insertion into an ear canal of a passenger to reduce sound levels within a travel environment of the transport vehicle.

7. The personal sanitation travel kit of claim 4, further comprising:
an inflatable neck pillow to facilitate sleep and discomfort while confined to the occupant seating space of the transport vehicle, the inflatable neck pillow having a inflatable U-shaped bladder, a removable cover having an inner space dimensioned contain the inflatable U-shaped bladder.

8. The personal sanitation travel kit of claim 7, the inflatable neck pillow further comprising:
an access slot in the removable cover dimensioned to receive the inflatable U-shaped bladder within the inner space; and
a first cooperating fastener to selectively open and close the access slot.

9. The personal sanitation travel kit of claim 8, the inflatable neck pillow further comprising:
a second cooperating fastener at a terminal end the cover, the cooperating fastener operable to secure the inflatable neck pillow about a neck of the passenger.

10. The personal sanitation travel kit of claim 4, wherein the mini roll of multi-ply toilet paper further comprises:
a compressed roll of the multi-ply toilet paper wrapped without an inner core insert, wherein the multi-ply toilet paper is wrapped at a minimal radius.

11. The personal sanitation travel kit of claim 10, wherein the minimal radius is a negligible radius.
